(19) United States
(12) Patent Application Publication
(10) Pub. No.: US 2016/0276085 A1
(43) Pub. Date: Sep. 22, 2016

(54) VARIABLE HARDNESS ACTUATOR

(71) Applicant: OLYMPUS CORPORATION, Tokyo (JP)

(72) Inventors: Kaoru MATSUKI, Kawasaki-shi (JP); Yu KONDO, Yamato-shi (JP)

(73) Assignee: OLYMPUS CORPORATION, Tokyo (JP)

(21) Appl. No.: 15/171,000

(22) Filed: Jun. 2, 2016

Related U.S. Application Data
(63) Continuation of application No. PCT/JP2014/079233, filed on Nov. 4, 2014.

(30) Foreign Application Priority Data
Dec. 3, 2013 (JP) 2013-250454

(51) Int. Cl. H01F 13/00; A61B 1/005; H01F 7/06

(52) U.S. Cl. CPC H01F 13/003; H01F 7/064; A61B 1/0057

(57) ABSTRACT

A variable hardness actuator includes a flexible tubular member; plural particles of magnetic powder filling the tubular member; a coil wound around an outer circumference of the tubular member, surrounding the magnetic powder; a drive unit configured to supply electric current to the coil; and a directing unit used to give a direction to the drive unit to supply the electric current, wherein the coil generates a magnetic field using the electric current supplied from the drive unit in response to the direction from the directing unit, causing the plural particles of magnetic powder to magnetize spontaneously, magnetically couple together, and harden.

### CROSS REFERENCE TO RELATED APPLICATION

This application is a continuation application of PCT/JP2014/079233 filed on Nov. 4, 2014 and claims benefit of Japanese Application No. 2013-250454 filed in Japan on Dec. 3, 2013, the entire contents of which are incorporated herein by this reference.

### BACKGROUND OF THE INVENTION

1. Field of the Invention

The present invention relates to a variable hardness actuator capable of changing rigidity against bending force.

2. Description of the Related Art

For example, when a tube is inserted into a complex-shaped lumen, if the tube is flexible, upon hitting a bent shape portion of the lumen, the tube deforms and stays in place, making it difficult to further insert the tube deep into the lumen.

Thus, a variable hardness actuator which can change rigidity of a member against a bending force has conventionally been proposed, and used, for example, in the field of endoscopes to improve ease of insertion.

As an example of such a variable hardness actuator, Japanese Patent No. 5124629 describes a hardness adjusting apparatus configured to change a compression state of a close wound coil spring by pulling a wire attached to a distal end of the close wound coil spring and thereby change hardness. Furthermore, Japanese Patent No. 5124629 describes a technique for reducing a force using an elastic body provided in a wire pulling mechanism, by taking into consideration the fact that a large force is required for an operation of pulling the wire. Specifically, the technique is designed to shift traction torque needed for pulling, toward a negative side, using a torsion spring or spiral spring as an elastic body, but because there is a portion in which the traction torque becomes negative, action of a negative tractive force is restrained using a combination of a worm gear and worm wheel.

### SUMMARY OF THE INVENTION

A variable hardness actuator according to an aspect of the present invention includes: a tubular member having flexibility; a plurality of magnetic bodies filling the tubular member; a coil wound around an outer circumference of the tubular member, surrounding the magnetic bodies; a drive unit configured to supply electric current to the coil; and a directing unit used to give a direction to the drive unit to supply the electric current, wherein the coil generates a magnetic field using the electric current supplied from the drive unit in response to the direction from the directing unit, causing the plurality of magnetic bodies to magnetize spontaneously, magnetically couple together, and harden.

### BRIEF DESCRIPTION OF THE DRAWINGS

FIG. 1 is a diagram showing a configuration of a variable hardness actuator according to a first embodiment of the present invention;

FIG. 2 is a diagram showing a state of the variable hardness actuator when hardened, in the first embodiment;

FIG. 3 is a time chart showing how hardness of the variable hardness actuator changes when an applied current is changed, in the first embodiment;

FIG. 4 is a diagram showing a configuration of a variable hardness actuator according to a modification of the first embodiment;

FIG. 5 is a diagram showing a state of a variable hardness actuator when not hardened, in a second embodiment of the present invention;

FIG. 6 is a diagram showing a state of the variable hardness actuator when hardened, in the second embodiment;

FIG. 7 is a diagram showing a state of a variable hardness actuator when not hardened, in a third embodiment of the present invention;

FIG. 8 is a diagram showing a state of the variable hardness actuator when hardened, in the third embodiment;

FIG. 9 is a diagram showing a configuration of a variable hardness actuator according to a modification of the third embodiment; and

FIG. 10 is a diagram showing a configuration of a variable hardness actuator according to a fourth embodiment of the present invention.

### DETAILED DESCRIPTION OF THE PREFERRED EMBODIMENTS

Embodiments of the present invention will be described below with reference to the drawings.

### First Embodiment

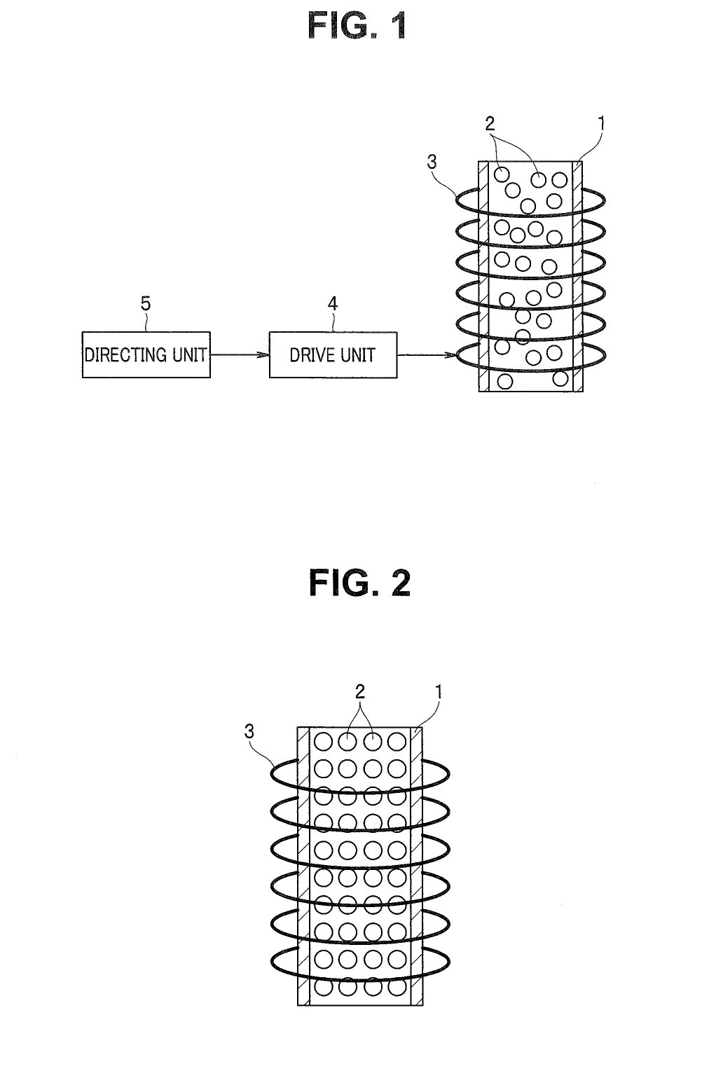

FIGS. 1 to 4 show a first embodiment of the present invention, where FIG. 1 is a diagram showing a configuration of a variable hardness actuator.

As shown in FIG. 1, the variable hardness actuator includes a tubular member 1, magnetic powder 2, a coil 3, a drive unit 4, and a directing unit 5.

The tubular member 1 is a tube-shaped member provided with flexibility and configured to flex when a bending force is applied. Note that it is assumed below that the tubular member 1 has (but is not limited to) a cylindrical shape, in particular. Then, an equal amount of bending is available when a bending force is applied in any direction perpendicular to an axial direction (radial direction of the cylinder) as long as a magnitude of the force is the same.

The magnetic powder 2 is powdered magnetic bodies, and the tubular member 1 is filled with plural particles of the magnetic powder 2. Note that FIG. 1 shows the variable hardness actuator in a non-hardened state and the magnetic powder 2 is randomly distributed in the tubular member 1. In this state, the variable hardness actuator can bend flexibly under a bending force applied in any direction perpendicular to an axial direction of the tubular member 1.

The coil 3 is an electrically conductive coil wound around an outer circumference of the tubular member 1, surrounding a portion filled with the magnetic powder 2.

The drive unit 4 supplies electric current to the coil 3, and includes, for example, a power source. The drive unit 4 according to the present embodiment is further able to control an amount of the electric current (current value) to be supplied.

The directing unit 5, which is used to direct the drive unit 4 to supply electric current, is made up of, for example, operation switches and the like and can be operated lightly without requiring a special amount of physical strength. The directing unit 5 according to the present embodiment further enables specifying the amount of the electric current (current value) to be supplied by the drive unit 4.

Next, FIG. 2 is a diagram showing a state of the variable hardness actuator when hardened. Note that the drive unit 4 and directing unit 5 are omitted from illustration in FIG. 2 and each of subsequent figures as appropriate.

As the drive unit 4 supplies an electric current to the coil 3 in response to a direction from the directing unit 5, the coil 3 generates a magnetic field. The generated magnetic field causes the magnetic powder 2 to magnetize spontaneously and thereby magnetically couple together. The magnetic coupling here is not used in a narrow sense of approaching, coming into contact, and getting concatenated by magnetic force, but means that particles of the magnetic powder 2 interact one another through magnetism (including action at a distance) (thus, for example, magnetic repulsion whereby N poles or S poles keep away from each other is also included in the magnetic coupling).

Thus, when a magnetic field is generated, the magnetic powder 2 freely movable in absence of a magnetic field gets clustered, for example, by being arranged along magnetic lines of force generates resistance friction in a direction orthogonal to the magnetic field, thereby gets united, increases in rigidity against external bending forces (especially a bending force applied in a direction perpendicular to the axial direction, such as described above), and hardens the variable hardness actuator.

Here, one of the reasons why plural magnetic bodies in a magnetic field harden is considered to be approximately as follows.

That is, when a magnetic body is placed in a magnetic field, spontaneous magnetization occurs as described above. The spontaneously magnetized magnetic body magnetically interact with other magnetic bodies moves to such a position (stable point) as to minimize energy of an entire system. In contrast, when one attempts to move the magnetic body from the stable point to another position (hereinafter referred to as an unstable point) by applying an external force (e.g., bending stress), energy corresponding to a difference value in potential energy of the magnetic field between the stable point and unstable point is required for that. Then, a force tending to return to the stable point, a force resisting external forces, is produced on the magnetic body located at the unstable point. Thus, it is considered that a bending stress needed in order to produce same bending deformation as in absence of a magnetic field becomes larger in presence of a magnetic field, increasing rigidity against the bending stress and resulting in a greater hardness.

Also, another one of the reasons why plural magnetic bodies in a magnetic field hardens is frictional forces produced when the plural magnetic bodies are brought into contact by magnetic force as described above.

Note that when behavior of the plural magnetic bodies are described figuratively, the magnetic bodies can be said to be fluidal in the absence of a magnetic field because individual magnetic bodies can move independently of one another, but can be said to be solid like in the presence of a magnetic field because motion of any one of the magnetic bodies affect motion of all the other magnetically coupled magnetic bodies (see also a fourth embodiment described later).

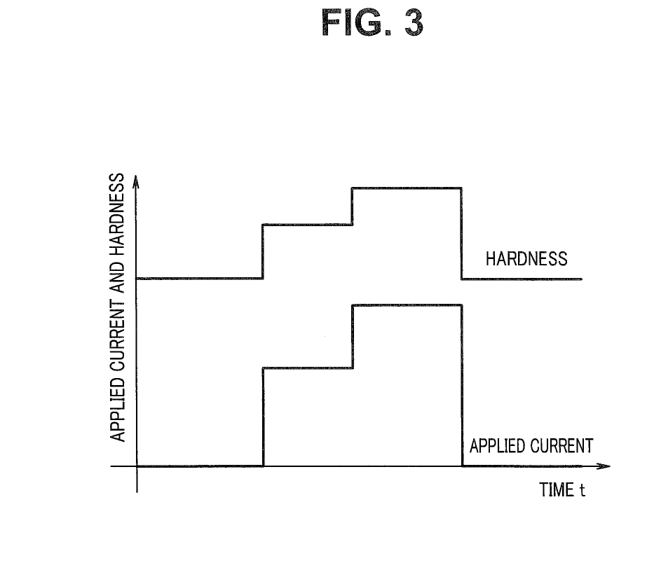

Next, FIG. 3 is a time chart showing how hardness of the variable hardness actuator changes when an applied current is changed.

The variable hardness actuator according to the present embodiment can adjust hardness by controlling the amount of electric current supplied to the coil 3.

To begin with, even when no electric current is supplied, the variable hardness actuator has some hardness depending on the type, wall thickness, and wire diameter of material making up each member.

Next, the larger the amount of electric current supplied to the coil 3, the stronger the magnetic force generated by the coil 3. Consequently, the spontaneous magnetization of magnetic powder 2 becomes stronger, i.e., the magnetic coupling among the particles of the magnetic powder 2, and thus the frictional forces acting on the magnetic powder 2 become stronger. In this way, the hardness of the variable hardness actuator increases according to the amount of electric current supplied to the coil 3.

Thus, by controlling the amount of electric current supplied to the coil 3, the variable hardness actuator according to the present embodiment can adjust hardness of the plural particles of the magnetic powder 2 and thus the hardness of the variable hardness actuator itself.

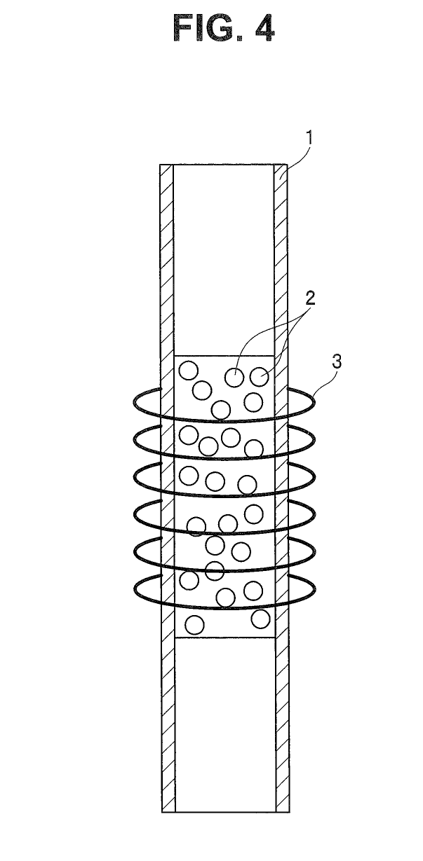

Next, FIG. 4 is a diagram showing a configuration of a variable hardness actuator according to a modification.

In the variable hardness actuator shown in FIG. 4, the magnetic powder 2 and coil 3 are placed only in part of the tubular member 1 in an axial direction. With adoption of this configuration, if the magnetic powder 2 and coil 3 are placed only in a desired part, hardness can be changed only in the desired part.

Note that by placing the magnetic powder 2 over almost an entire area of the tubular member 1 in the axial direction and placing plural coils 3 at different locations in the axial direction, the plural coils 3 may be allowed to be supplied with electric currents independently of one another. In so doing, the amounts of the supplied electric current may be further configured to be able to be controlled independently among the plural coils 3. It is difficult to change hardness only in a desired part even if a configuration such as described above is adopted because magnetic fields leak out of the coil 3 in the axial direction, but because the magnetic field weakens with a distance from the coil 3 supplied with electric current, it is possible to cause changes in hardness by centering around the desired part.

The first embodiment configured as described above makes it possible to generate a magnetic field by applying electric current to the coil 3, cause the magnetic bodies (magnetic powder 2 in the present embodiment) in the tubular member 1 to couple together magnetically through spontaneous magnetization, and thereby stably change the hardness of the magnetic bodies and thus the variable hardness actuator. In so doing, since frictional forces act among particles of the magnetic powder 2, higher hardness is available.

Also, since the hardness of the variable hardness actuator can be adjusted through electric control, namely by controlling the amount of electric current, operation can be performed via the directing unit 5 using operation switches and the like without requiring a special amount of physical strength. Also, abrasion, metal fatigue, and the like which will occur if a mechanical configuration is adopted can be reduced.

Furthermore, when the magnetic bodies and coil 3 are placed in part of the tubular member 1 in an axial direction, the hardness in the locations of the magnetic bodies and coil 3 alone can be changed as desired.

Besides, since the magnetic powder 2 is used as a magnetic body, an operation of thoroughly filling the tubular member 1 can be performed easily. In so doing, since the magnetic powder 2 can move freely, the hardness can be changed smoothly according to magnetic field strength.

Thus, the variable hardness actuator according to the present embodiment allows an operation of changing hardness to be carried out lightly without relying on the amount of physical strength.

### Second Embodiment

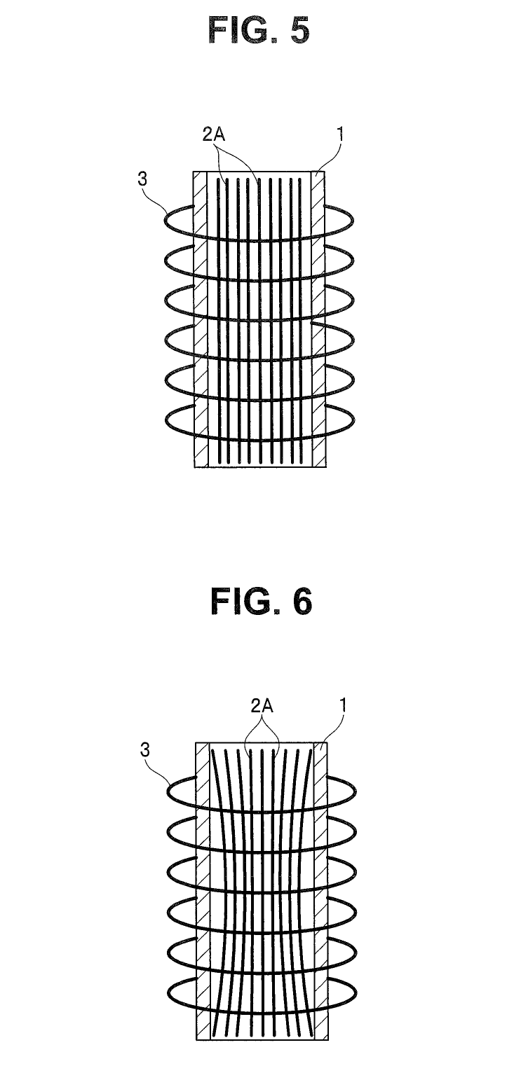

FIGS. 5 and 6 show a second embodiment of the present invention, where FIG. 5 is a diagram showing a state of a variable hardness actuator when not hardened and FIG. 6 is a diagram showing a state of the variable hardness actuator when hardened.

In the second embodiment, description of parts similar to those of the first embodiment described above will be omitted as appropriate by taking measures such as denoting the parts with the same reference numerals as the corresponding parts and mainly differences from the first embodiment will only be described.

Whereas in the first embodiment described above, the magnetic bodies are made up of magnetic powder 2, in the present embodiment, the magnetic bodies are made up of magnetic wire rods 2A.

That is, the magnetic bodies according to the present embodiment are bendable magnetic wire rods 2A. A plurality of the magnetic wire rods 2A are placed along the axial direction of the tubular member 1 and filled into the tubular member 1. Axial length of the magnetic wire rods 2A is almost equal to or a little shorter than a filling region in the tubular member 1.

Next, when an electric current is supplied to the coil 3, a generated magnetic field causes the magnetic wire rods 2A to magnetize spontaneously and function in a manner approximately similar to a bar magnet extending in the axial direction. Then, as shown in FIG. 6, the magnetic wire rods 2A are arranged, for example, by taking a state of being flexed along lines of magnetic force as a stable point and coupled together magnetically. In so doing, even if an external force (e.g., bending stress) is applied, a force tending to return to the stable point is produced on the magnetic wire rods 2A, increasing rigidity against the bending stress and resulting in hardening.

With this configuration, the hardness of the variable hardness actuator can be changed by controlling the amount of electric current supplied to the coil 3, as with the first embodiment described above.

The second embodiment configured as described above provides advantages approximately similar to those of the first embodiment described above. Besides, the second embodiment which uses the magnetic wire rods 2A as magnetic bodies is free from spreading of the magnetic bodies unlike powder and makes an operation of filling the tubular member 1 easier. Also, the magnetic wire rods 2A provides the advantage of being easier to handle than powder even before being filled into the tubular member 1.

### Third Embodiment

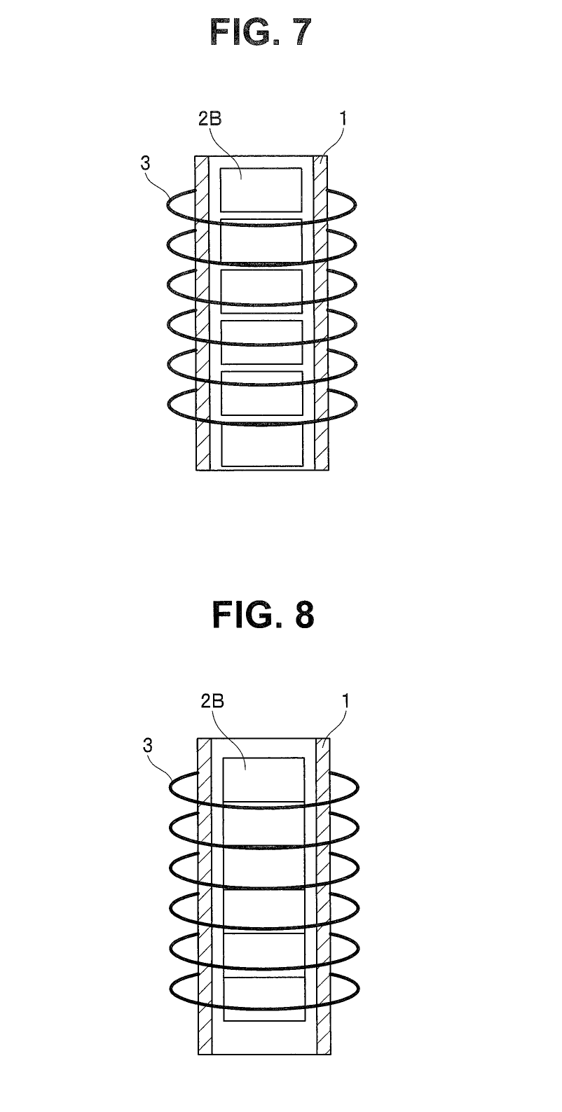

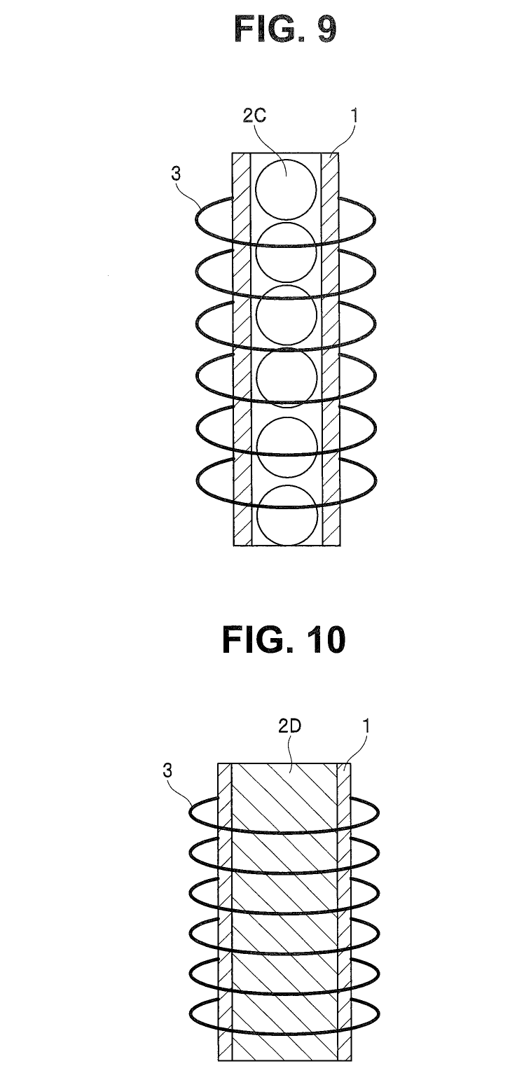

FIGS. 7 to 9 show a third embodiment of the present invention, where FIG. 7 is a diagram showing a state of a variable hardness actuator when not hardened, FIG. 8 is a diagram showing a state of the variable hardness actuator when hardened, and FIG. 9 is a diagram showing a configuration of a variable hardness actuator according to a modification.

In the third embodiment, description of parts similar to those of the first and second embodiments described above will be omitted as appropriate by taking measures such as denoting the parts with the same reference numerals as the corresponding parts and mainly differences from the first and second embodiments will only be described.

The present embodiment uses columnar magnetic bodies 2B or spherical magnetic bodies 2C as magnetic bodies.

To begin with, the magnetic bodies shown in FIGS. 7 and 8 are columnar magnetic bodies 2B with an outside diameter a little smaller than an inside diameter of the cylindrical tubular member 1. The plural columnar magnetic bodies 2B are arranged in a line, with a central axis of the columnar magnetic bodies 2B placed along the axial direction of the tubular member 1, i.e., the columnar magnetic bodies 2B are arranged such that a top face of one columnar magnetic body 2B and a bottom face of another columnar magnetic body 2B will be opposed to each other.

With this configuration, when no electric current is supplied to the coil 3, movements of the plural columnar magnetic bodies 2B are not specially related to one another as shown in FIG. 7 and bendability of the flexed variable hardness actuator is rarely disturbed.

In contrast, when an electric current is supplied to the coil 3, a generated magnetic field causes each of the columnar magnetic bodies 2B to magnetize spontaneously and thereby become a columnar bar magnet. Furthermore, the plural columnar magnetic bodies 2B come into contact and get concatenated by magnetic coupling to function in a manner approximately similar to a single bar magnet extending in the axial direction.

At this time, due to static frictional forces produced on contact surfaces, by forces of attraction among the columnar magnetic bodies 2B acting as normal forces, the concatenated plural columnar magnetic bodies 2B behave as a single unified object as a whole, increase in rigidity, and become hardened.

Then, because forces of attraction, and thus maximum static frictional forces, among the columnar magnetic bodies 2B can be changed by controlling the amount of electric current supplied to the coil 3, the hardness of the variable hardness actuator can be adjusted as with the first and second embodiments described above.

Note that although the columnar magnetic bodies 2B with a short columnar shape are illustrated in FIGS. 7 and 8, to ensure bendability (bendability whereby shape changes smoothly without becoming stepwise) of the variable hardness actuator when no magnetic field is generated, thinner disk-shaped columnar magnetic bodies 2B can be adopted (this means that the number of columnar magnetic bodies 2B placed in the tubular member 1 will be increased).

Next, magnetic bodies shown in FIG. 9 are spherical magnetic bodies 2C with a diameter a little smaller than the inside diameter of the tubular member 1, and a plurality of the spherical magnetic bodies 2C are arranged in a line along the axial direction of the tubular member 1.

Operation resulting from use of the spherical magnetic bodies 2C is approximately similar to operation resulting from use of the columnar magnetic bodies 2B, but due to the spherical shape, the magnetic bodies contact each other via point contact rather than surface contact.

Thus, when the spherical magnetic bodies 2C are used as magnetic bodies, because of a small contact area, hardness may fall slightly compared to when the columnar magnetic bodies 2B are used if a same amount of electric current is supplied to the coil 3, but on the other hand, the variable hardness actuator can be made easier to bend and a maximum bendable angle can be increased.

Note that although the columnar magnetic bodies 2B and spherical magnetic bodies 2C are cited as examples here, it is advisable to use magnetic bodies of an appropriate shape by taking into consideration a hardening efficiency which represents what degree of hardness is available when a certain amount of electric current is supplied, a maximum bendable angle, smoothness of bending, and the like. Magnetic bodies of other shapes may be adopted, including, for example, a magnetic body with circular contact surfaces obtained by slightly cutting opposite ends of a spherical body in a diameter direction of the spherical body and a magnetic body of a shape obtained by bonding bases of two congruent truncated right circular cones.

The third embodiment configured as described above provides advantages approximately similar to those of the first and second embodiments described above. Besides, when the columnar magnetic bodies 2B are used as magnetic bodies, upon being placed in a magnetic field, the columnar magnetic bodies 2B come into contact with one another in a central axis direction, forming one long bar magnet, and can harden into a solid state. Also, because of a large contact area among the columnar magnetic bodies 2B, the hardness of the hardened magnetic bodies can be increased. Furthermore, the columnar magnetic bodies 2B are advantageous in being able be filled into the tubular member 1 and handled before the filling more easily than powder. Also, the plural columnar magnetic bodies 2B arranged in the axial direction are divided into sections rather than individual items being continuous in the axial direction as in the case of the magnetic wire rods 2A, even if the columnar magnetic bodies 2B are subjected to a force in a direction perpendicular to the axial direction when no magnetic field is applied, repulsion resulting from elasticity of material does not occur. Thus, the hardness of the variable hardness actuator can be kept low when no electric current is supplied to the coil 3, and thus a dynamic range of hardness change can be increased.

On the other hand, use of the spherical magnetic bodies 2C as magnetic bodies is advantageous in that ease of bending can be improved and the maximum bendable angle can be increased compared to when the columnar magnetic bodies 2B are used.

### Fourth Embodiment

FIG. 10 is a diagram showing a fourth embodiment of the present invention and showing a configuration of a variable hardness actuator.

In the fourth embodiment, description of parts similar to those of the first to third embodiments described above will be omitted as appropriate by taking measures such as denoting the parts with the same reference numerals as the corresponding parts and mainly differences from the first to third embodiments will only be described.

The present embodiment uses a viscous magnetic fluid 2D as a magnetic body.

That is, according to the present embodiment, the tubular member 1 is filled with a viscous magnetic fluid 2D called MRF (magneto rheological fluid) and plural magnetic bodies according to the present embodiment are plural fine magnetic particles (preferably fine ferromagnetic particles) dispersed uniformly in the viscous magnetic fluid 2D.

Specifically, the viscous magnetic fluid 2D is a magnetic colloidal solution made up of a base liquid such as water or oil, fine ferromagnetic particles of magnetite, manganese, zinc ferrite, or the like, and a surface-active agent covering surfaces of the fine ferromagnetic particles and having affinity for the base liquid. The fine ferromagnetic particles here have submicroscopic size, for example, with a diameter of around 10 nm. Also, the surface-active agent covering the surfaces of the fine ferromagnetic particles has affinity for the base liquid as described above, but the surface-active agent is dispersed homogeneously in the base liquid without aggregation and sedimentation because of repulsive forces acting between molecules of the surface-active agent and thereby maintains a stable colloidal state.

It is known that when the viscous magnetic fluid 2D configured as described above is subjected to a magnetic field, the fine magnetic particles form clusters, for example, along lines of magnetic force (e.g., spiking phenomenon), generate resistance friction in a direction orthogonal to the magnetic field, and exhibit a property (i.e., Bingham fluid-like property) of behaving as a solid up to a certain yield stress (the yield stress depends on magnetic field strength), but behaving as a fluid with shearing stress increasing in proportion to shearing velocity when subjected to a stress equal to or higher than the yield stress.

Thus, when a magnetic field is generated by supplying electric current to the coil 3, the variable hardness actuator can be hardened. In so doing, by controlling the amount of electric current supplied to the coil 3, the hardness of the variable hardness actuator can be changed as with the first to third embodiments described above.

The fourth embodiment configured as described above provides advantages approximately similar to those of the first to third embodiments described above. Besides, since the viscous magnetic fluid 2D is used, when subjected to a magnetic field, the fine magnetic particles dispersed in colloidal form in a fluid move more freely than in powder form and always provide same hardness if the amount of electric current is the same, making it possible to smoothly change hardness with high reproducibility.

Note that the present invention is not limited to the precise embodiments described above and may be embodied by changing components in the implementation stage without departing from the spirit of the invention. Also, various aspects of the invention can be implemented using appropriate combinations of the components disclosed in the above embodiments. For example, some of the components disclosed in the embodiments may be deleted. Furthermore, components may be combined as required across different embodiments. Thus, needless to say that various alterations and applications are possible without departing from the spirit of the invention.

What is claimed is:

1. A variable hardness actuator comprising:
a tubular member having flexibility;
a plurality of magnetic bodies filling the tubular member;
a coil wound around an outer circumference of the tubular member, surrounding the magnetic bodies;
a drive unit configured to supply electric current to the coil; and
a directing unit used to give a direction to the drive unit to supply the electric current, wherein
the coil generates a magnetic field using the electric current supplied from the drive unit in response to the direction from the directing unit, causing the plurality of magnetic bodies to magnetize spontaneously, magnetically couple together, and harden.

2. The variable hardness actuator according to claim 1, wherein:
the drive unit is able to control an amount of the electric current supplied;
the directing unit allows the amount of the electric current supplied by the drive unit to be specified; and
hardness of the plurality of magnetic bodies is adjustable by controlling the amount of the electric current supplied to the coil.

3. The variable hardness actuator according to claim 2, wherein the magnetic bodies and the coil are placed in part of the tubular member in an axial direction.

4. The variable hardness actuator according to claim 2, wherein the magnetic bodies are magnetic powder.

5. The variable hardness actuator according to claim 2, wherein the magnetic bodies are bendable magnetic wire rods placed along an axial direction of the tubular member.

6. The variable hardness actuator according to claim 2, wherein the magnetic bodies are columnar magnetic bodies having an outside diameter smaller than an inside diameter of the tubular member, and a plurality of the columnar magnetic bodies are arranged in a line, with a central axis of the columnar magnetic bodies being placed along an axial direction of the tubular member.

7. The variable hardness actuator according to claim 2, wherein the magnetic bodies are spherical magnetic bodies having a diameter smaller than an inside diameter of the tubular member, and a plurality of the spherical magnetic bodies are arranged in a line along an axial direction of the tubular member.

8. The variable hardness actuator according to claim 2, wherein the plurality of magnetic bodies are a plurality of fine magnetic particles dispersed in a fluid making up a viscous magnetic fluid filling the tubular member.
